(19) United States
(12) Patent Application Publication
(10) Pub. No.: US 2006/0211012 A1
(43) Pub. Date: Sep. 21, 2006

(54) MHC CLASS II AS A PREVENTIVE VACCINE AGAINST HIV INFECTION

(75) Inventor: Geoffrey W Hoffmann, Vancouver (CA)

Correspondence Address: VERMETTE & CO., BOX 40, GRANVILLE SQUARE, SUITE 230 - 200 GRANVILLE STREET, VANCOUVER, BC, V6C 1S4, CA

(73) Assignee: NETWORK IMMUNOLOGY INC., Vancouver, BC (CA)

(21) Appl. No.: 11/307,951

(22) Filed: Feb. 28, 2006

Related U.S. Application Data
(60) Provisional application No. 60/594,231, filed on Mar. 21, 2005.

(51) Int. Cl. C12Q 1/70; C12Q 1/68

(52) U.S. Cl. 435/6; 435/5

(57) ABSTRACT

A preventive vaccine for AIDS, suitable for HIV negative persons, is described.

This application claims the benefit of previously filed Provisional Patent Application Ser. No. 60/594,231, filed on Mar. 21, 2005.

### FIELD OF THE INVENTION

The present invention concerns a vaccine that protects people who are HIV negative from becoming infected with HIV. HIV is human immunodeficiency virus. HIV causes acquired immunodeficiency syndrome (AIDS) in people infected with the virus.

### BACKGROUND TO THE INVENTION

All publications mentioned hereunder are incorporated herein by reference.

A vaccine that prevents infection with HIV is urgently needed. Many approaches have been tried and failed. The classical approaches involve inducing immunity to HIV proteins.

SIV is simian immunodeficiency virus, a virus that is similar to HIV, and causes an AIDS-like disease in macaque monkeys. It has been shown in an experiment with two macaques that protection against SIV infection can result from immunity to human MHC class II. Arthur et al. 1995 J. Virol. 69, 3117-3124.

### BRIEF DESCRIPTION OF THE DRAWING

Further features and advantages will be apparent from the following Detailed Description of the Invention, given by way of example, of a preferred embodiment taken in conjunction with the accompanying drawing, wherein:

FIG. 1: This figure shows a model of HIV pathogenesis, which underlies the invention. Helper T cells are selected to have some complementarity to MHC class II. Suppressor T cells are selected such that their V regions have complementarity to as many helper T cell V regions as possible. In addition to CD4, CXCR4 and CCR5, the T cell receptor of HIV-specific helper T cells is a coreceptor for HIV. HIV variants that are recognized by the largest number of helper T cells are preferentially produced, and also stimulate these helper T cells to proliferate. In other words, there is co-selection of helper T cells and HIV variants. There is also co-selection of helper T cells and suppressor T cells. Since HIV and the suppressor T cells are subject to the same selection criterion, there is a natural selection process in which HIV proteins and the V regions of the suppressor T cells converge in shape space. Anti-HIV immunity then becomes specific for the suppressor T cell quasi-species. Immunity against this central regulating element of the system results in the collapse of the system. The suppressor T cells no longer adequately stimulate and regulate the helper T cells, that are then free to inappropriately help immune responses against self components, and autoimmunity ensues. The autoimmunity includes immunity directed against helper T cells that express CD4.

### DETAILED DESCRIPTION OF THE INVENTION

Much evidence supports the concept that AIDS is an autoimmune disease triggered by HIV. Kion et al. 1991, Science, 253, 1138-1140; Bourinbaiar et al. 2005, Autoimmunity Reviews 4, 403-409. A model of AIDS pathogenesis explains how the interaction of HIV with the immune system results in the collapse of a central regulating component of the T cell repertoire, leading to autoimmunity. Hoffmann 1994, Immunol. and Cell Biol., 72, 338-346; Hoffmann, 1995, Scand. J. Immunol., 41, 331-337.

Experiments have shown that xenoimmunity and alloimmunity can be protective against HIV infection and SIV infection. Stott et al. 1991, Nature 353, 393; Spear et al. 2001, J. Acquir. Immune Defic. Syndr. 26, 103-110; Wang et al. 1999, Nature Medicine, 5, 1004-1009; MacDonald et al. 1998, J. Infect. Dis. 177, 551-556; Peters et al. 2004, Lancet, 363, 518-524; Leith et al. 2003, Aids Res. Hum. Retrovir. 19, 957-965. These experiments lead to a new interpretation of the model of Hoffmann, 1994, op. cit, which is the basis of the present invention. A review of that model follows.

A “quasi-species” is a diverse population of similar elements. The term was originally coined to refer to related macromolecules in an origin of life scenario. Eigen et al. 1988, J. Phys. Chem. 92, 6881-6891. HIV is a rapidly mutating virus, and a consequently diverse array of HIV virions can be referred to as a quasispecies.

“Co-selection” refers to mutual selection of members within two diverse populations, as the result of some sort of complementarity in shapes of members of one of the populations to shapes in the other population. Hoffmann 1994, Immunol. and Cell Biol., 72, 338-346.

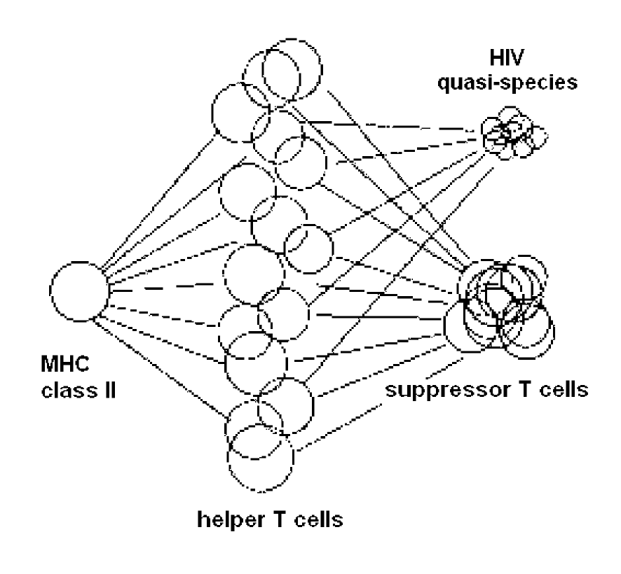

Helper T cells are highly diverse, but are nevertheless generally selected to have some affinity for MHC class II. In the model shown in FIG. 1 there are suppressor T cells that are selected according to the criterion of being able to recognise and be idiotypically stimulated by as many helper T cells as possible. The suppressor T cell population is then a quasi-species, with the similarity in its idiotypes being the result of the uniform selection criterion for all of these cells. There is co-selection of helper T cells and the suppressor T cells.

A key postulate of the model is that the antigen-specific helper T cell receptor is a coreceptor for infection by HIV. Hoffmann, op. cit 1994. This means that helper T cells that specifically recognise the virus are preferentially infected, and these helper T cells are also stimulated by HIV to proliferate. This postulate has been validated. Douek et al. 2002 Nature 417, 95-98. Strains of HIV that are recognised by the largest number of helper T cells preferentially replicate. We then have co-selection also of HIV and helper T cells.

HIV and the suppressor T cell population are then subject to the same selection pressure, namely to recognise as many helper T cell idiotypes as possible. The consequence in many cases is that the HIV quasi-species and the suppressor cell quasi-species converge in shape space, meaning that the average shape of the HIV population looks more and more like the average shape of the suppressor T cell idiotypes. At the same time there is anti-HIV immunity. This immunity then cross-reacts with the idiotypes of the suppressor T cell population, and this centrally regulating element of the system comes under attack from the anti-HIV immunity. The helper cells are then no longer adequately regulated, and autoimmunity ensues. The model is directly supported by some remarkable findings that link autoimmunity, alloimmunity, idiotypic network regulation and AIDS. Kion et al. 1991, op cit. Autoimmunity in AIDS includes cytotoxic T cell activity against CD4 helper T cells. Grant et al. 1996, Immunol. and Cell Biol. 74, 38-44.

The conclusion that was previously derived from this scenario was that we should endeavour to eliminate HIV-specific T cells. Hoffmann, 1995, op. cit. That may be extremely difficult, given the diversity of the virus and the diversity of the helper T cells. Surprisingly, a new interpretation leads to a very different preventive vaccine design, which is the subject of the present invention.

Helper T cells that recognise both HIV and suppressor cell idiotypes play a key role in the convergent selection process. When HIV infection occurs, those clones are especially selected, and they lead to the dominant selection of HIV variants and suppressor cell clones that resemble each other. The average shape of the HIV quasi-species thus undergoes selection in virus carriers to resemble the average shape of the suppressor T cell idiotypes. If however the average shape of the suppressor T cell idiotypes in an individual differs sufficiently from the average shape of the HIV with which an individual is infected, the population of helper T cells with which HIV interacts may be effectively distinct from the population interacting with the suppressor T cells. Then there are two separate co-selection processes, that do not necessarily converge.

Live xenogeneic cells and allogeneic cells are potent antigens. Hence xenoimmunization and alloimmunization cause substantial changes to the T cell and B cell repertoires. The average shape of the specific receptors of the helper T cell population changes, or moves in shape space, with the consequence that the average shape of the specific receptors of the coselected suppressor cell population moves in shape space. This shift can be sufficient, such that the infecting HIV then interacts with a population of helper T cells distinct from the population with which the suppressor T cells interact. The convergence of HIV and the suppressor cell population then does not occur.

### PREFERRED EMBODIMENTS

The vaccine does not need to contain live cells or dead cells. The main component in the vaccine, that causes the required shift in the T cell repertoires, is foreign MHC class II molecules. The foreign MHC class II can be xenogeneic MHC class II or an altered version of human or xenogeneic MHC class II. Allogeneic MHC class II molecules that are present in some members of the population are not preferred, because for a given human MHC class II molecule, some individuals will normally have that molecule as a self antigen, and hence immunization of those individuals with it will not cause the required shift.

In a preferred embodiment the vaccine contains xenogeneic MHC class II molecules from a species that is more phylogenetically different from both humans and macaque monkeys, than humans are phylogenetically different from macaque monkeys. For example, the vaccine can consist of mouse MHC class II. The immune response of a macaque to mouse MHC class II is similar to that of a human to mouse MHC class II. This vaccine prevents both SIV infection in macaques and HIV infection in humans. The same agent can therefore go directly from an animal trial in macaques to clinical trials in humans. This enables rapid development of the vaccine.

An advantage of using mouse MHC class II is that the immune response of a human to mouse MHC class II is similar to the immune response of a mouse to human MHC class II, since the phylogenetic distance is exactly the same in both cases. Parameters of immune responses in mice to immunizations with human MHC class II are therefore predictive of parameters for converse immunizations of humans with mouse MHC class II. This advantage is not shared by chemically modified MHC class II. Furthermore, the mouse and human immune systems are the two systems that have been most studied most extensively by immunologists.

The MHC class II vaccine can be given with or without an adjuvant. The possible need for an adjuvant can be assessed, by one skilled in the art, for example in experiments in which mice are immunized with human MHC class II.

MHC class II molecules are hetero-dimers, each consisting of an α and a β chain. In one embodiment of the invention multimers of these heterodimers are used. Multimers are more effective in cross-linking T cell receptors and are therefore more immunogenic than the simple MHC class II heterodimers. For example, MHC class II tetramers are available, that have been produced for other purposes.

In one embodiment of the invention the MHC class II molecules are aggregated to make them more immunogenic. The need for such aggregation can be assessed by one skilled in the art, for example in experiments in which mice are immunized with human MHC class II.

The MHC class II molecules need to be different from the MHC class II of the immunized person. In addition to xenogeneic or allogeneic MHC class II, a chemically modified version of MHC class II can be effective. There is a practically unlimited number of ways in which MHC class II can be chemically modified.

This vaccine is strictly for people who are not already infected with HIV. It will not be effective for people who are already infected. Furthermore, giving it to HIV positive people may result in the emergence of HIV strains against which the preventive vaccine is ineffective. The vaccine will therefore be given to individuals only after they have been given an HIV test, and have been shown to be HIV negative.

The suppressor T cells that were described mainly in the 1970s and 1980s included Ts1, Ts2 and Ts3 cells. In the context of a model of Ts1, Ts2 and Ts3 cells formulated by Germain et al., Scand. J. Immunol. 13, 1-10, the more recently described CD4+ regulatory T cells, Sakaguchi et al. 1995, J. Immunol., 155, 1151-1164; McGeachy et al. J. Immunol. 175, 3025-3032, can be most simply interpreted as being Ts1 cells, while the suppressor T cells of FIG. 1 are Ts2 cells.

What is claimed is:

1. A vaccine for the prevention of HIV infection, in which said vaccine contains MHC class II molecules.

2. The vaccine of claim 1, in which said MHC class II molecules are multimers.

3. The vaccine of claim 1 or 2, in which said MHC class II molecules are tetramers.

4. The vaccine of claims 1 to 3, in which the MHC class II molecules are non-human MHC class II molecules.

5. The vaccine of claims 1 to 4, in which the MHC class II molecules are from a species that is more phylogenetically distant from humans than humans are phylogenetically distant from macaque monkeys.

6. The vaccine of claims 1 to 5, in which the MHC class II molecules are mouse MHC class II molecules.

7. The vaccine of claims 1 to 6, in which the MHC class II molecules are in aggregated form.

8. The vaccine of claims 1 to 7, in which the vaccine additionally contains an adjuvant.

9. The vaccine of claims 1 to 8, in which the MHC class II molecules are chemically modified.

10. A method for the prevention of HIV infection, comprising administering to an individual a vaccine containing MHC class II molecules.

11. The method of claim 10, in which said MHC class II molecules are multimers.

12. The method of claim 10 or 11, in which said MHC class II molecules are tetramers.

13. The method of claims 10 to 12, in which the MHC class II molecules are non-human MHC class II molecules.

14. The method of claims 10 to 13, in which the MHC class II molecules are from a species that is more phylogenetically distant from humans than humans are phylogenetically distant from macaque monkeys.

15. The method of claims 10 to 14, in which the MHC class II molecules are mouse MHC class II molecules.

16. The method of claims 10 to 15, in which the MHC class II molecules are in aggregated form.

17. The method of claims 10 to 16, in which the vaccine additionally contains an adjuvant.

18. The method of claims 10 to 17, in which the MHC class II molecules are chemically modified.
